(19) United States
(12) Patent Application Publication
(10) Pub. No.: US 2019/0125971 A1
(43) Pub. Date: May 2, 2019

(54) SMALL VOLUME TISSUE PROCESSING DEVICES

(71) Applicant: LifeCell Corporation, Madison, NJ (US)

(72) Inventors: Nathaniel Bachrach, Clifton, NJ (US); Kai-Roy Wang, Jersey City, NJ (US); Evan J. Friedman, West Orange, NJ (US)

(21) Appl. No.: 16/171,774

(22) Filed: Oct. 26, 2018

Related U.S. Application Data
(60) Provisional application No. 62/577,949, filed on Oct. 27, 2017.

(51) Int. Cl. A61M 5/178; A61M 5/31; A61L 27/36

(52) U.S. Cl. CPC A61M 5/1782; A61M 5/3145; A61M 5/3129; A61L 27/3604; A61L 27/3687; A61M 1/0072; A61L 27/3695; A61M 2202/08; A61M 2005/3128; A61M 2205/8206; A61L 27/3691

(57) ABSTRACT

The present disclosure provides systems and methods for processing small volumes of tissue. The systems and methods include an interior fill volume and an exterior wash volume separated by a filter. Waste and fluids pass through the filter during tissue washing and transfer and can be absorbed or removed by application of negative pressure. The systems and methods are more portable and require fewer transfer steps than conventional methods, thus simplifying tissue processing procedures.

This application claims priority to U.S. Provisional Application No. 62/577,949, filed Oct. 27, 2017, the entire contents of which is incorporated herein by reference.

The present disclosure relates to devices and methods for treatment of relatively small volumes of tissue, particularly adipose tissue.

Fat grafting, including autologous grafting, has become increasingly common and has numerous clinical applications such as facial contouring, breast reconstruction and/or augmentation, and other aesthetic or reconstructive procedures. In addition, autologous fat grafting has been found to have relatively low donor-site morbidity compared with other surgical options.

Autologous fat grafting is a popular procedure in small-volume applications such as facial scarring, lip augmentation, and facial rhytids (particularly in otherwise difficult-to-address areas such as the nasolabial fold and glabellar furrows).

In some cases, however, autologous fat grafting provides somewhat unpredictable outcomes. For example, the amount of adipose cell viability after implantation is variable, which can result in less than optimal outcomes and/or require multiple or revision procedures.

Adipocyte viability can be affected by a number of factors including aspiration pressure, injection pressure, and shear stress. If done improperly, the loading and unloading of cells from syringes and other vessels can result in damage to the cells and reduce overall cell viability after implantation. To mitigate these effects, the user must exert careful control over pressures and shear stresses when loading and unloading cells. This control can be achieved by introducing a level of automation and repeatability in cell transfer.

Further, adipose tissue transfer generally requires one or more steps wherein adipose tissue is passed between tissue collection, processing, or delivery devices. These steps can be time-consuming. In addition, the transfer steps, including loading of vessels or syringes, can cause a reduction in cell viability if undesirably large forces (e.g., shear forces) are placed on tissues during the process.

The present disclosure provides devices and methods for improved tissue transfer particularly in the realm of small volumes. The devices and methods have improved portability and can reduce operative times while also reducing the number of transfer steps needed in conventional systems.

A system for treating tissue is provided according to various embodiments described herein. The system includes a syringe body that encloses a volume. The system includes a filter disposed in the syringe body and dividing the volume into an interior fill volume and an exterior wash volume. The filter is configured to allow passage of fluids. The system includes a cannula coupled to the syringe body. Applying a negative pressure to the syringe body draws a tissue through the cannula and into the interior fill volume and transfers fluid between the interior fill volume and the exterior wash volume.

A method for treating tissue is provided according to various embodiments described herein. The method includes selecting a syringe body that encloses a volume and a filter disposed in the syringe body and dividing the volume into an interior fill volume and an exterior wash volume. The filter is configured to allow passage of fluids. A cannula is coupled to the syringe body. The method also includes drawing a tissue through the cannula and into the interior fill volume by applying a negative pressure. The method also includes transferring fluid from the tissue through the filter to the exterior wash volume by applying the negative pressure. The method may include injecting the tissue at an implantation site.

### BRIEF DESCRIPTION OF THE DRAWINGS

FIG. 1 illustrates a system for treating tissue in accordance with various embodiments of the present disclosure.

FIG. 2 illustrates the system of FIG. 1 during aspiration in accordance with various embodiments of the present disclosure.

FIG. 3 illustrates the system of FIG. 1 during compression in accordance with various embodiments of the present disclosure.

FIG. 4 illustrates a canister system for treating tissue in accordance with various embodiments of the present disclosure.

FIG. 5 illustrates a system for treating tissue including a mixing turbine in accordance with various embodiments of the present disclosure.

FIGS. 6A-6C illustrate steps of a tissue treatment procedure in accordance with various embodiments of the present disclosure.

FIG. 7 illustrates a perspective view of a system for treating tissue attached to a portable vacuum source in accordance with various embodiments of the present disclosure.

FIG. 8 illustrates a cross-sectional view of the vacuum source of FIG. 7.

FIG. 9 illustrates a system integrally attached to a pump for treating tissue in accordance with various embodiments of the present disclosure.

FIGS. 10A and 10B illustrate a tubing system for treating tissue in accordance with various embodiments of the present disclosure.

FIG. 11 illustrates a system for treating tissue including a turbine and an auger in accordance with various embodiments described herein.

### DESCRIPTION OF CERTAIN EXEMPLARY EMBODIMENTS

Reference will now be made in detail to certain exemplary embodiments according to the present disclosure, certain examples of which are illustrated in the accompanying drawings. Wherever possible, the same reference numbers will be used throughout the drawings to refer to the same or like parts.

In this application, the use of the singular includes the plural unless specifically stated otherwise. In this application, the use of “or” means “and/or” unless stated otherwise. Furthermore, the use of the term “including,” as well as other forms such as “included” and “includes,” is not limiting.

The use of the word “syringe” is not limited to any industry standard and includes any of a variety of receptacles in different shapes and sizes. Any range described herein will be understood to include the endpoints and all values between the endpoints.

As used herein, “tissue processing” can refer to a number of steps or treatments intended to clean or process tissue. Such steps can include washing, removal of collagen strands, mechanical agitation or separation, or removal or filtration of waste and wash from harvested tissue.

As used herein, “adipose tissue” refers to adipose tissue obtained by any means including, for example, liposuction and/or tumescent liposuction. In addition, the adipose tissue may be substantially intact or may be altered by, for example, washing with saline, antimicrobials, detergents, or other agents; the addition of therapeutic agents such an analgesics, antimicrobials, and anti-inflammatories; the removal of some cells or acellular components; or disruption or alteration by the collection process itself including, for example, during liposuction or tumescent liposuction. The adipose tissue can be autologous tissue, allogeneic tissue, or xenogenic tissue (e.g., porcine tissue).

As used herein, “small volumes” generally refers to volumes of the order of 100 mL or less.

The section headings used herein are for organizational purposes only and are not to be construed as limiting the subject matter described. All documents, or portions of documents, cited in this application including but not limited to patents, patent applications, articles, books, and treatises are hereby expressly incorporated by reference in their entirety for any purpose.

Various human and animal tissues can be used to produce products for treating patients. For example, various tissue products have been produced for regeneration, repair, augmentation, reinforcement, and/or treatment of human tissues that have been damaged or lost due to various diseases and/or structural damage (e.g., from trauma, surgery, atrophy, and/or long-term wear and degeneration). Fat grafting, including autologous fat grafting, can be useful for a variety of clinical applications including facial fillers, breast augmentation, buttock augmentation/sculpting, augmentation of other tissue sites, correction of lumpectomy defects, cranial-facial defect correction, and correction of lipoplasty defects (divots).

Autologous fat grafting is a popular procedure in small-volume applications such as facial scarring, lip augmentation, and facial rhytids (particularly in otherwise difficult-to-address areas such as the nasolabial fold and glabellar furrows). Systems and methods described herein allow for processing of small volumes of tissue during harvest or transfer. By reducing the time to process tissue and combining or removing steps needed in conventional processing systems, systems and methods disclosed herein allow performance of small-scale procedures with less set-up time and less operative time. In addition, systems and methods described herein include little “dead space” where tissue can be lost in a conventional system. Each transfer or processing step generally reduces the volume of viable tissue at the end of the procedure due to losses (e.g., tissue trapped on filters or stuck in tubes that cannot be removed). By reducing the number of operations needed to process tissue, a greater amount of recoverable tissue is created by the systems and methods described herein.

In addition, loading of injection devices or otherwise transferring tissue prior to implantation or during processing can be time consuming. Accordingly, the present disclosure provides devices and methods that assist in loading/unloading of tissue transfer devices, thereby reducing operative time. In some embodiments, loading and unloading steps are eliminated or reduced as the tissue is processed in place or during transit from the patient to a filling device. In some embodiments, the systems, devices, and methods can be used to transfer adipose tissues or other implantable materials (e.g., injectable or implantable gels, pastes, or putties).

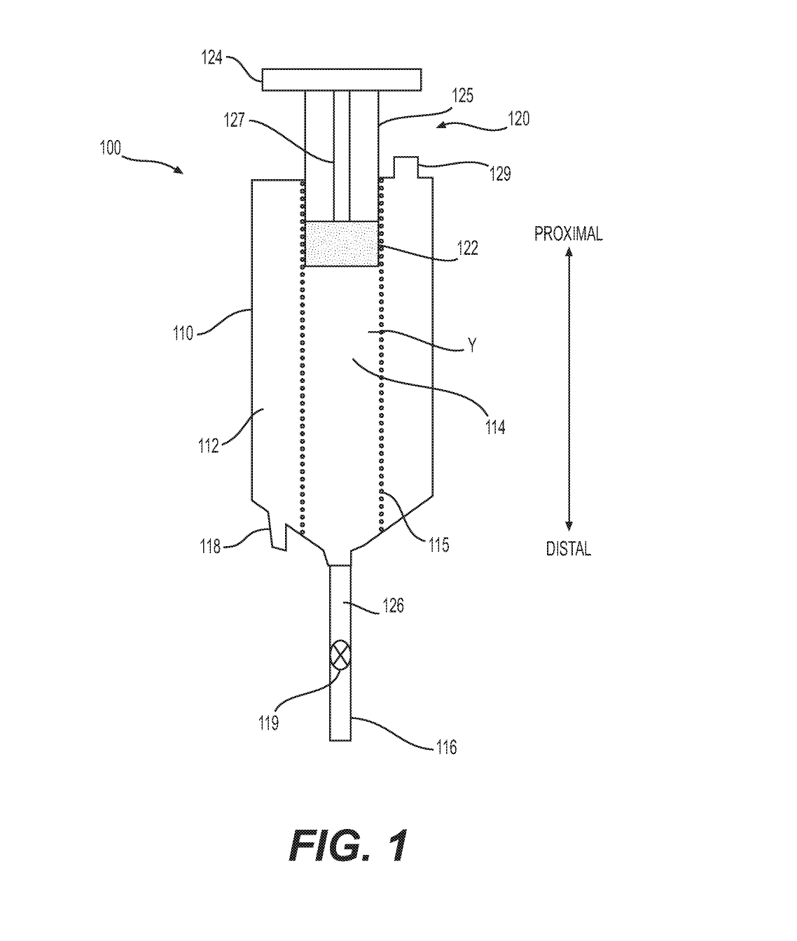

FIG. 1 illustrates a system 100 for treating tissue in accordance with various embodiments of the present disclosure. The system 100 can include a syringe body 110 enclosing a volume, a filter 115, and a cannula 116 coupled to the syringe body 110. The filter 115 is disposed in the syringe body 110 and divides the volume into an interior fill volume 114 and an exterior wash volume 112. When a negative pressure is applied to the syringe body 110, tissue can be drawn through the cannula 116 and into the interior fill volume 114, and fluid can be transferred between the interior fill volume 114 and the exterior wash volume 112 through the filter 115. Once the tissue is cleaned within the system 100, the tissue can be injected using a plunger 120. The system 100 can be used for aspiration, cleaning, and injection of small volumes of tissue in a single device without requiring a number of additional transfer steps through tubes among multiple devices.

The syringe body 110 can be a portion of a commercially available syringe that is adapted into the system 100 or can be formed specifically for use with the system 100 according to various embodiments. In some embodiments, the volume enclosed by the syringe body 110 can be less than 100 mL. Compared to canister-based adipose processing products, smaller volume devices allow for a high degree of portability and ensure adequate mixing and cleaning of the tissue within the volume. In various embodiments, negative pressure can be applied to the syringe body 110 by withdrawing the plunger 120 (e.g., pulling back on a base 124 of the plunger 120). In some embodiments, the exterior wash volume 112 can be three to four times larger than the interior fill volume 114. In some embodiments, the volume of the exterior wash volume 112 can be sufficiently large as to accommodate enough wash solution from an initial stage that no further wash solution need be introduced during a processing operation.

In some embodiments, the filter 115 can allow fluids to pass through while preventing passage of tissue components such as cells. The filter 115 can be formed of a variety of materials. In some embodiments, the filter 115 can be formed of a mesh material such as a porous polymer mesh or metal mesh. In some embodiments, the filter 115 can be a screen or netting. In some embodiments, the filter 115 can be designed to allow oil flow and/or to reduce collagen adsorption that can lead to pore clogging. The filter 115 can be rigid or pliable in various embodiments. The holes or pores in the filter 115 can be approximately 200 μm in diameter or less than 200 μm in diameter in various embodiments. The filter 115 can be optimized in some embodiments to separate waste from lipoaspirate.

In some embodiments, the filter 115 can have a fine pore size to isolate the stromal vascular fraction (SVF) from other waste products. The SVF can include a high concentration of preadipocytes, mesenchymal stem cells, or endothelial progenitor cells that could have the capacity to enable adipose tissue regeneration.

In some embodiments, the porous filter 115 can extend from the distal end of the syringe body all the way to the proximal end. In these embodiments, the plunger 120 can be equipped with a sheath 125 to block or seal the pores in the filter 115 and prevent leaking from the exterior wash volume 112 behind a stopper 122 of the plunger. In embodiments lacking a sheath 125, an O-ring or other sealing element can create a seal between a shaft 127 of the plunger 120 and the proximal end of the syringe body 110.

In some embodiments, a portion of the filter 115 can be porous while another portion can be non-porous. For example, the filter 115 can be porous distal to point Y in FIG. 1 but non-porous proximal to point Y. In such embodiments, the plunger 120 of the system 100 can be sized so that the stopper 122 does not extend distal to point Y during operation of the device. This arrangement can prevent fluid from escaping behind the stopper 122 of the plunger 120. In some embodiments, the point Y can be about one-quarter, one-third, one-half, two-thirds, or three-quarters of the way from the distal end to the proximal end of the syringe body.

In some embodiments, the filter 115 can be cylindrical and can cause the interior fill volume 114 and the exterior wash volume 112 to be positioned concentrically. In some embodiments, the exterior wash volume 112 can form an annular ring that wraps around the interior fill volume 114 or vice versa.

In some embodiments, the cannula 116 can have a sharpened end that can penetrate into the patient. The cannula 116 provides a lumen 126 to transfer tissue into the interior fill volume 114 of the syringe body 110. In some embodiments, the cannula 116 can have a large gauge or diameter such as 3.0 mm to provide a wide lumen 126 and prevent tissue from clogging the lumen.

The cannula 116 can include a valve 119 that may be closed to block the lumen 126 and prevent passage therethrough. In some embodiments, the valve 119 can be manually operated. In some embodiments, the valve 119 can be a one-way or check valve that allows tissue to enter the syringe body 110 through the lumen 126 but prevents tissue from exiting. Although the valve 119 is shown on the cannula 116 in FIG. 1, it is also possible to place the valve in the syringe body 110 near the point of attachment of the cannula 116.

In some embodiments, a port 118 can be located in fluid communication with the exterior wash volume 112. The port 118 can be used to introduce or withdraw fluids into the volume enclosed by the syringe body. For example, the port 118 can be attached to a vacuum source such as a portable or non-portable vacuum pump, house vacuum in a laboratory or clinic, or a manual pump such as a bulb or spring-loaded pump. The vacuum source can draw fluid from the interior fill volume 114 to the exterior wash volume 112 through the filter 115 and finally out of the port 118. In some embodiments, the port 118 can include a valve to allow the port to be closed. Although one port 118 is shown in FIG. 1, additional ports may be located within or otherwise engaged with the syringe body 110 or passing through the plunger 120.

In some embodiments, the port 118 can be connected to a fluid source to provide cleaning solution into the exterior wash volume 112. The cleaning solution can include water, saline solution, lactated Ringer's solution, or any other appropriate solution to rinse, clean, or treat the tissue in the interior fill volume 114. The cleaning solution can also include additional factors such as salts, antibacterials, detergents, or buffers. In various embodiments, the cleaning solution can be forced into the exterior wash volume 112 by applying positive pressure at the fluid source or can be drawn into the exterior wash volume 112 by applying a negative pressure within the syringe body 110.

In some embodiments, the syringe body 110 can include a vent 129. In some embodiments, the vent 129 can allow air to pass through but not fluids or solids. The vent 129 can include a one-way or check valve that will only allow air to enter into the enclosed volume in the syringe body 110 and prevents air from exiting the volume. For example, the one-way valve can close in response to the application of positive pressure from inside the syringe body 110.

In some embodiments, the interior fill volume 114 of the syringe body 110 can be filled with tissue, such as fat, to be washed as described below. With the cannula 116 inserted into the tissue to be drawn in, the syringe plunger 120 is pulled back. The valve 119 in the cannula 116 is then closed and the port 118 is opened. The syringe plunger 120 is then pushed in to compress the interior fill volume 114. By so doing, excess fluid and/or poor-quality fat is pressed through the filter 115 into the exterior wash volume 112 where it is then evacuated through the port 118. The port 118 is then closed and the cannula valve 119 is reopened. The syringe plunger 120 is then pulled back again to bring additional tissue into the interior fill volume 114. This process can be repeated until the desired volume of tissue is held in the interior fill volume 114.

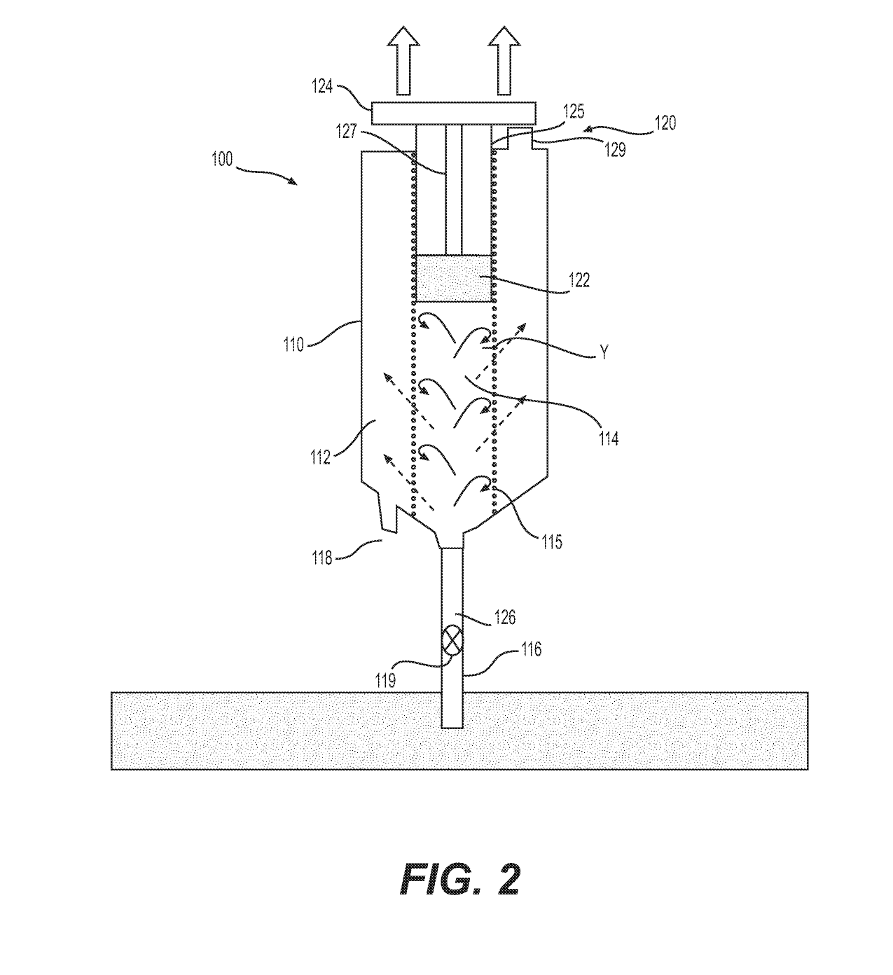

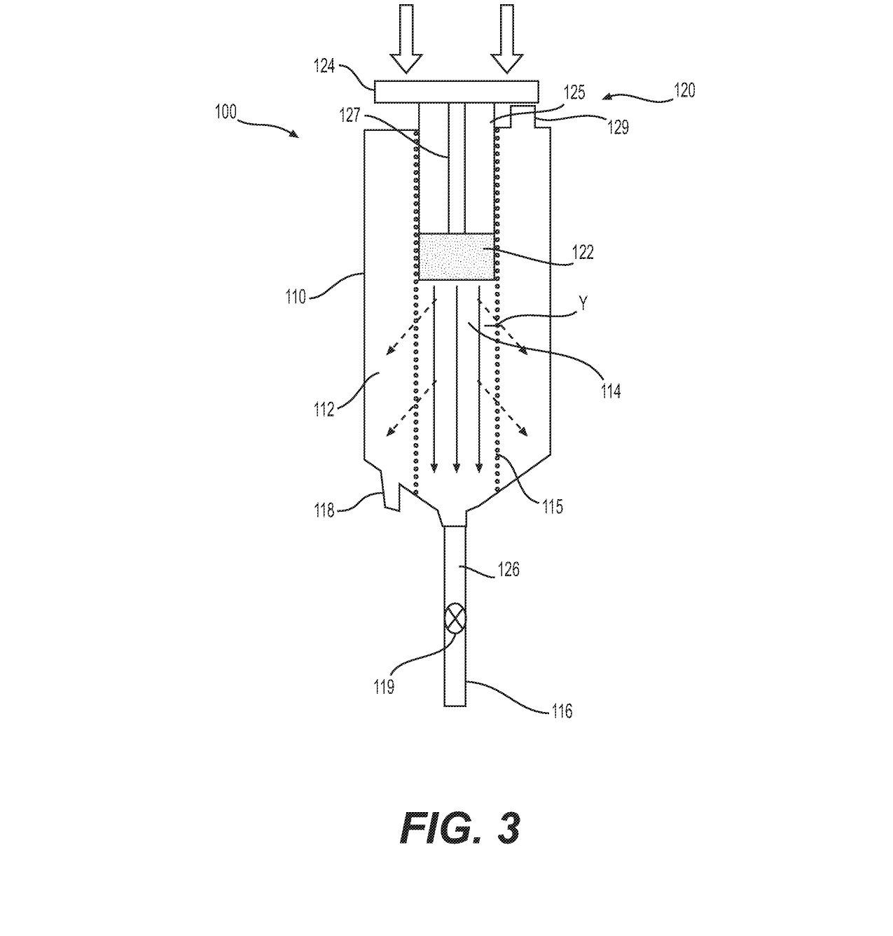

In some embodiments, the tissue can be cleaned or processed over a repeated series of steps as shown in FIGS. 2 and 3. As shown in FIG. 2, the tip of the cannula 116 is placed into a cleaning solution such as water, saline, or lactated Ringer's solution. The plunger 120 is pulled back to aspirate the solution into the interior fill volume 114. The inflow of cleaning solution mixes the tissue components and frees more of the impurities such as oil, blood, cell debris, or others from the tissue. The cannula valve 119 can then be closed and the port 118 can be opened. As shown in FIG. 3, the plunger 120 is then pushed in to compress the interior fill volume 114. This forces the cleaning solution to pass through the filter 115 and into the exterior wash volume 112. The cleaning solution carries with it all of the impurities while leaving the clean tissue inside the interior fill volume 114. During this process, some fluid can already begin to pass through the filter 115 and into the exterior wash volume 112. The cannula valve 119 can then be opened and the port 118 can be closed to allow repetition of this cycle until the tissue is washed to the desired degree.

In some embodiments, the washed tissue can be directly re-injected into the patient. For example, the cannula can pierce the skin of the patient at the injection site and the plunger 120 can be pressed in with the valve 119 of the cannula 116 open. In various embodiments, the same cannula can be used for re-injection as was used to draw up the tissue or the cannula can be replaced for safety.

In some embodiments, the cycles of tissue aspiration and tissue cleaning are entirely self-contained within the system 100. In other words, there is no need to transfer the tissue from device to device or through hoses or tubes. Thus, the tissue remains sterile or aseptic from withdrawal, throughout processing, and during re-injection. The self-contained system 100 need not be opened to air during the process in some embodiments.

Alternatively, a small-volume canister system can be used rather than a syringe system. The canister system is adapted for connection with a portable vacuum system to draw tissue and cleaning fluids into the system. Although the canister system can be equipped with a plunger to expel the tissue after cleaning, tissue is more often transferred out of the canister system to a separate syringe for re-injection.

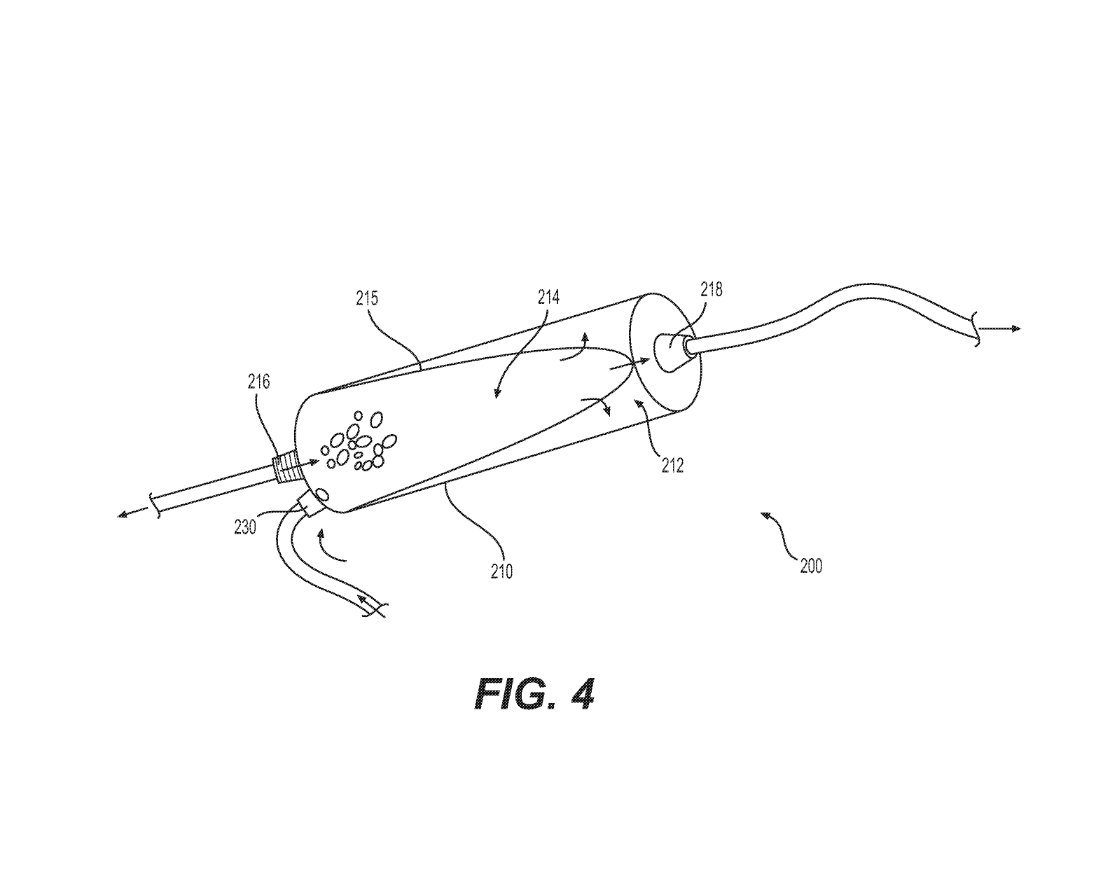

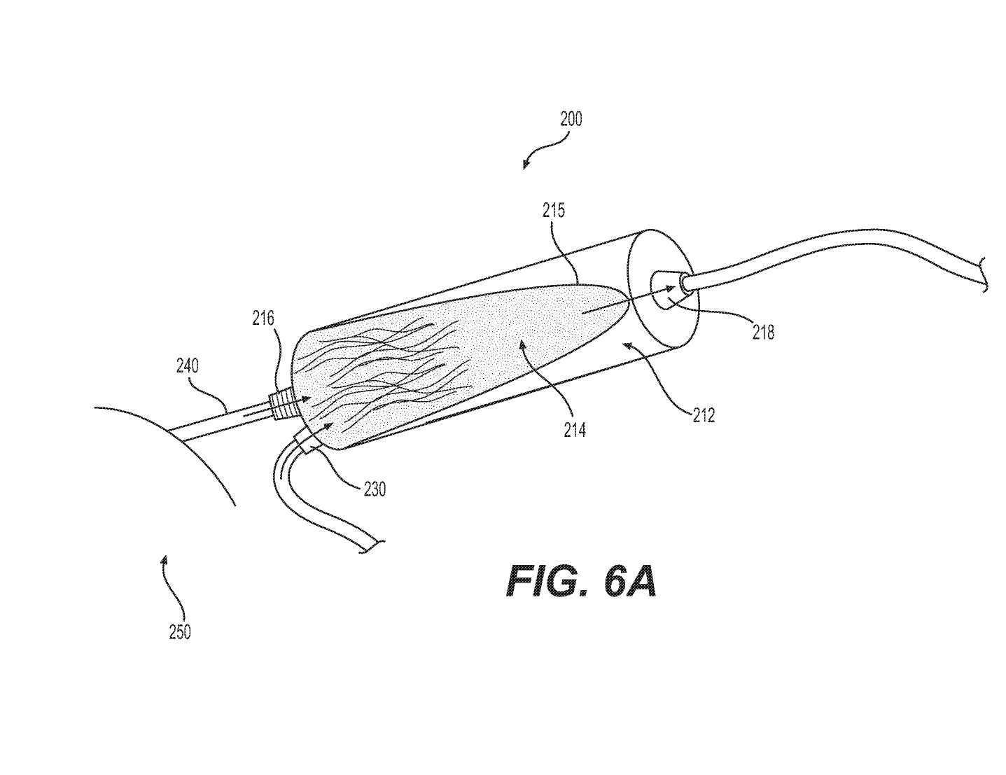

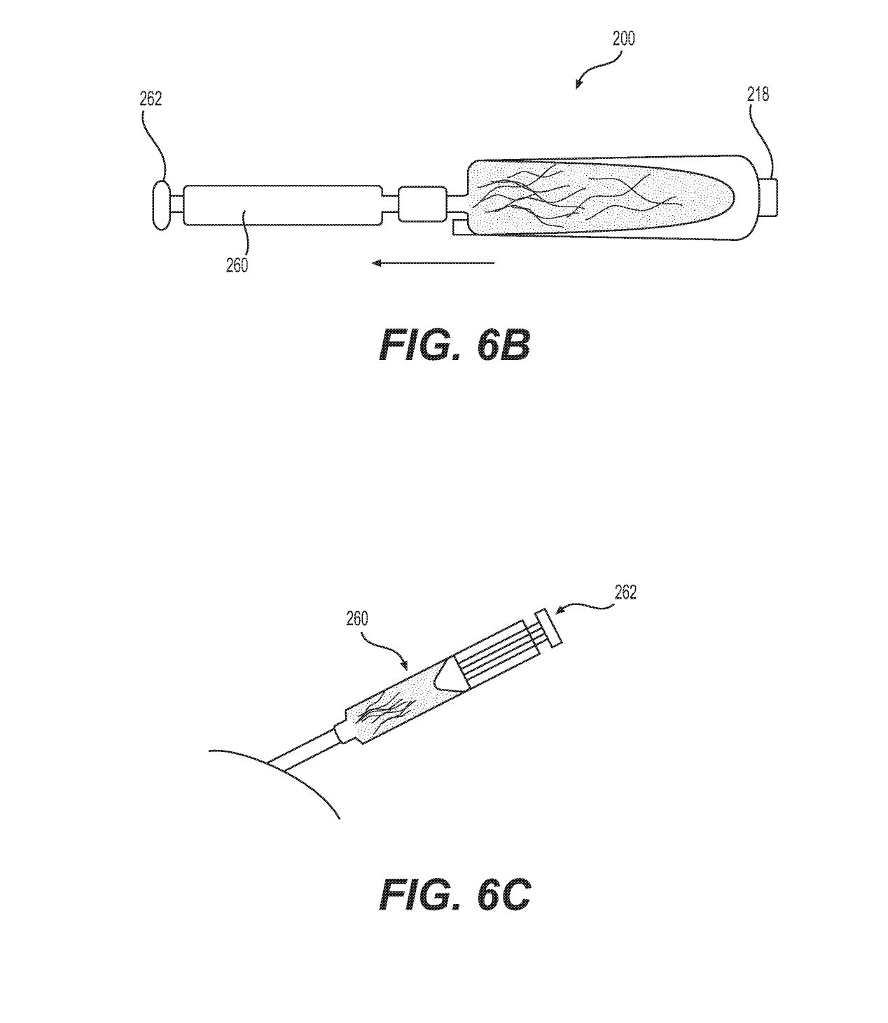

FIG. 4 illustrates a canister system 200 for treating tissue in accordance with various embodiments of the present disclosure. The system 200 can include a canister body 210 enclosing a volume, a filter 215, and a tissue port 216 couplable to a hose, tube, or cannula. The filter 215 is disposed in the canister body 210 and divides the volume into an interior fill volume 214 and an exterior wash volume 212. When a negative pressure is applied to the canister body 210 through a port 218, tissue can be drawn through the tissue port 216 and into the interior fill volume 214, and fluid can be transferred between the interior fill volume 214 and the exterior wash volume 212 through the filter 215. Once the tissue is cleaned within the system 200, it can be transferred to a syringe for re-injection as described in greater detail below with relation to FIGS. 6A-6C. The system 200 can allow simultaneous harvesting and washing of tissue and extraction of waste to result in clean adipose tissue that is ready for re-injection.

In accordance with various embodiments, the canister body 210 can include a port 230 that is connected to a fluid source including cleaning solution. The cleaning solution can be drawn into the exterior wash volume 212 or the interior fill volume 214 by the application of negative pressure at the port 218. In some embodiments, the system 200 can maintain a constant inflow of cleaning solution at port 230 and outflow of waste solution from port 218.

The port 230, port 218, and tissue port 216 can all include valves that may either be manually operated or one-way valves that open or close in response to certain pressure conditions within the canister body 210. For example, the tissue port 216 can be closed if the volume of tissue to be washed is within the interior fill volume 214 yet the vacuum pump at port 218 is still needed to draw fluid from port 230 and through the tissue and filter 215.

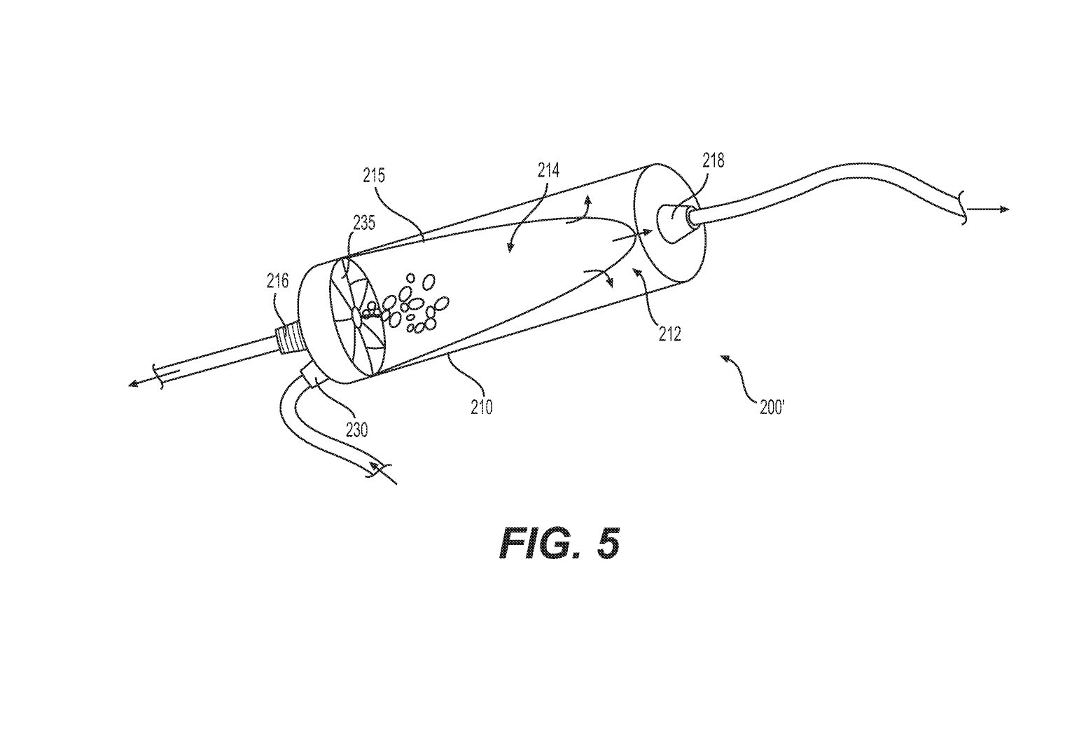

FIG. 5 shows an alternative embodiment of a canister system 200′ for treating tissue. The canister system 200′ is similar to the canister system 200 described above with reference to FIG. 4 but with the inclusion of a mixing turbine 235 near the port 230 and the port 216. In some embodiments, the mixing turbine 235 can increase the rate of flow of tissue through port 216 and/or the rate of fluid flow through port 230 into the interior fill volume 214. The mixing turbine 235 can apply physical force to the tissue to break up large sections of tissue or homogenize the tissue. In some embodiments, the mixing turbine 235 can itself provide agitation to the adipose tissue and can prevent clogging of the device. In some embodiments, the mixing turbine 235 can be used to capture larger collagen debris on paddles, shaft, or a comb of the turbine. The mixing turbine may be moved more distant from the ports 216, 230, and additional turbines 235 can be included within the system 200′.

In some embodiments, the canister systems 200, 200′ can include a plunger to facilitate removal of processed tissue from the canister body 210. For example, the end including the port 218 can be removable and replaced with a cap including a plunger. The plunger can then be depressed to press against the filter 215 and the tissue located in the interior fill volume 214. The tissue can then be ejected from the interior fill volume 214 through the port 216.

FIGS. 6A-6C illustrate steps in a procedure to process and transfer or re-inject tissue. In FIG. 6A, the port 216 of the canister system 200 is connected to a cannula 240 that is positioned in adipose tissue 250 within a patient. A negative pressure is applied at port 218 to cause tissue to be drawn through the cannula 240 and the tissue port 216 and into the interior fill volume 214. At the same time or subsequently, cleaning solution is drawn through port 230 and into the interior fill volume 214. Cleaning fluid picks up unwanted constituents of the tissue and becomes waste fluid. The waste fluid travels through the filter 215 and into the exterior wash volume 212 where it is drawn out of the system through the port 218.

In FIG. 6B, the processed tissue is transferred to a secondary syringe 260. In various embodiments, the transfer can be facilitated by withdrawing a plunger 262 of the secondary syringe 260 to pull material into the secondary syringe 260. Alternatively or in addition, a positive pressure can be applied at or near port 218 of the canister system 200. For example, positive air pressure can be applied at the port 218 or a plunger can be used to move the tissue as described above.

FIG. 6C illustrates the processed tissue being injected into the patient. For example, the plunger 262 of the secondary syringe 260 can be depressed to eject the tissue out of the secondary syringe 260.

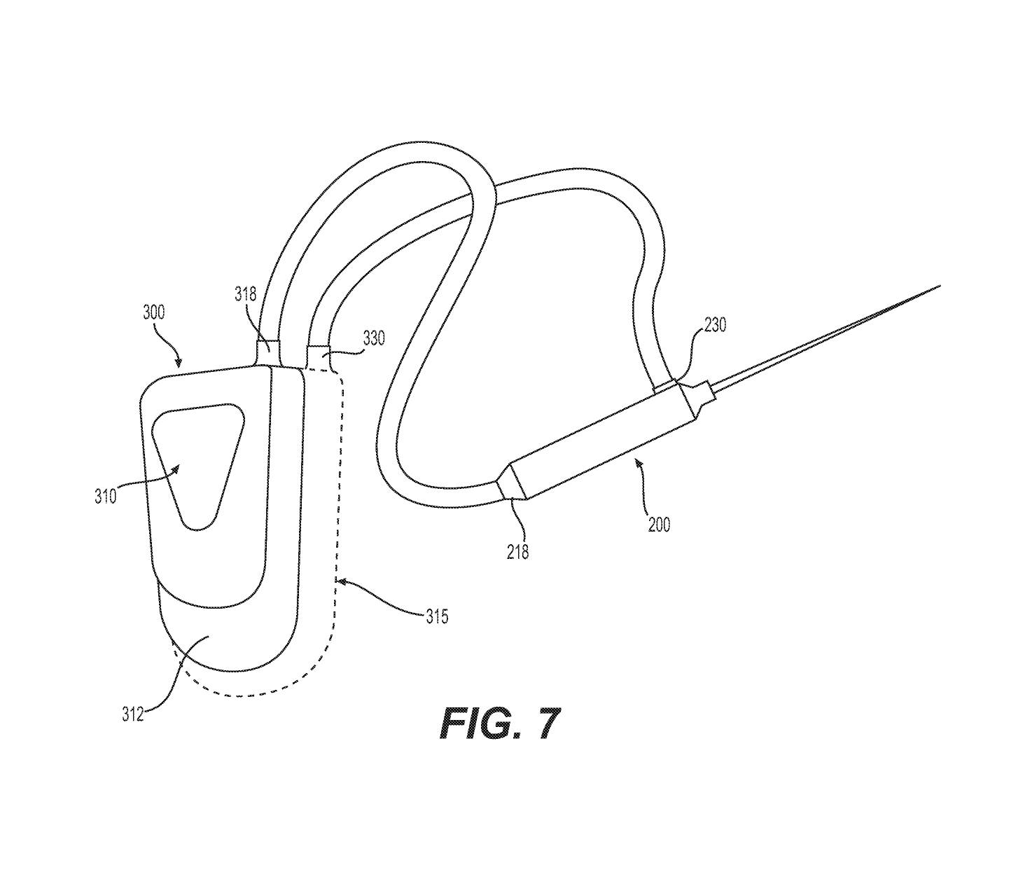

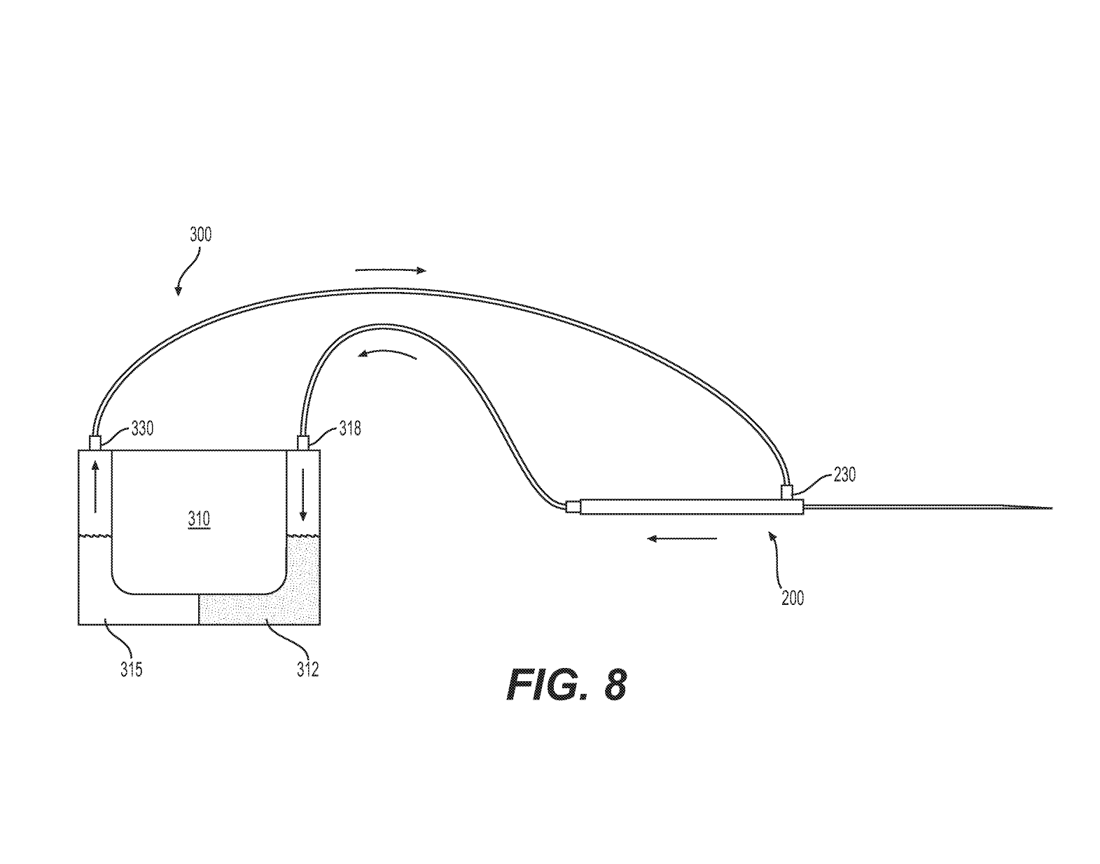

The present systems can also include additional components that further assist in maintaining portability and flexibility in use. For example, FIGS. 7 and 8 illustrate a perspective view and a cross-sectional view, respectively, of a portable vacuum source 300 attached to a canister system 200 for treating tissue in accordance with various embodiments of the present disclosure. The portable vacuum source 300 can include a battery-operated vacuum pump 310 and a waste container 312. In some embodiments, the portable vacuum source 300 can optionally include a wash fluid reservoir 315. The use of a portable vacuum source 300 allows for increased portability and simplicity in the system, and the structure of the portable vacuum source 300 can allow for simultaneous harvesting and washing to increase process efficiency and decrease total procedure time.

The waste container 312 can include a port 318 that is in fluid communication with the port 218 of the canister system 200. In some embodiments, port 318 and port 218 can be connected by a hose, tube, or other passageway including a lumen through which fluid may travel. The battery-operated vacuum pump 310 can draw waste fluid through port 318 and into the waste container 312. The waste container 312 can act as a reservoir to collect filtered waste and wash from the processed lipoaspirate in some embodiments.

In connection with the canister system 200, the battery-operated vacuum pump 310 can draw tissue into the interior fill volume 214 of the canister system 200. In some embodiments, the battery-operated vacuum pump 310 can include preset values of negative pressure that are chosen to prevent the application of excess shear stress to the tissue as it is drawn into the canister system 200. In some embodiments, the battery-operated vacuum pump 310 can include a peristaltic pump. The peristaltic pump can operate in a backwards or forwards direction and can have a controller to vary the speed. In some embodiments, the battery-operated vacuum pump 310 can be operated by a foot controller.

In optional embodiments, the portable vacuum device 300 can include a wash fluid reservoir 315 attached to or separate from the vacuum device. The wash fluid reservoir 315 can include a cleaning fluid such as saline or lactated Ringer's solution. In some embodiments, the wash fluid reservoir 315 can include a port 330. In some embodiments, the port 330 can be placed in fluid communication with the port 230 of the canister system 200. In such an embodiment, the application of negative pressure using the battery-operated vacuum pump 310 at port 218 can pull cleaning fluid from the wash fluid reservoir 315 through port 330 and port 230 and into the interior fill volume 214 of the canister system 200. In this manner, harvesting of tissue and washing of the tissue can be performed simultaneously.

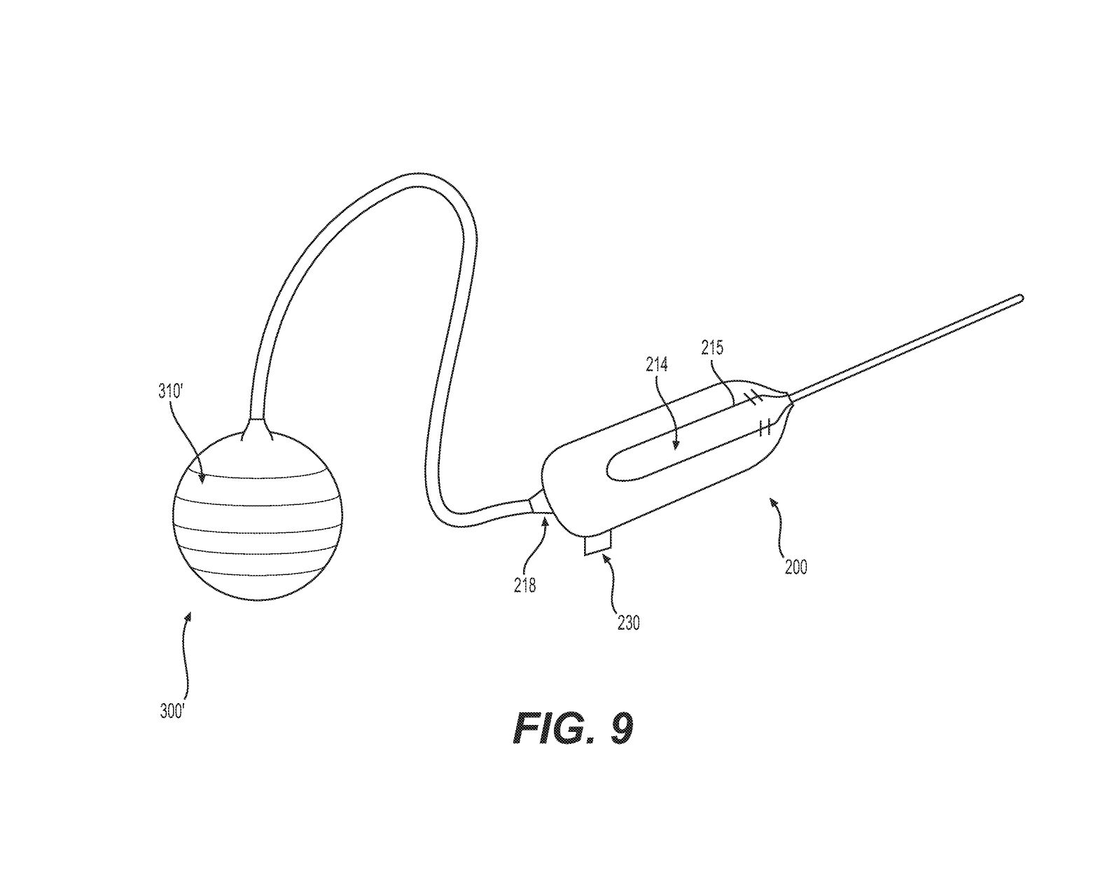

FIG. 9 illustrates an alternative embodiment of the portable vacuum system 300′ attached to a canister system 200 integrally attached to a pump for treating tissue in accordance with various embodiments of the present disclosure. The portable vacuum system 300′ can include a mechanically operated pump such as a bulb 310′ or spring-loaded pump. As described above with reference to FIGS. 7 and 8, the portable vacuum system 300′ can include a waste disposal feature incorporated into the system 300′. For example, in some embodiments, the bulb 310′ acts as both the vacuum source and the waste container. In other embodiments, the spring-loaded pump can include a waste container within space where vacuum is being drawn. Although bulb and spring-loaded pumps are described herein, one of ordinary skill in the art would appreciate that other mechanical pump arrangements are compatible with the present disclosure.

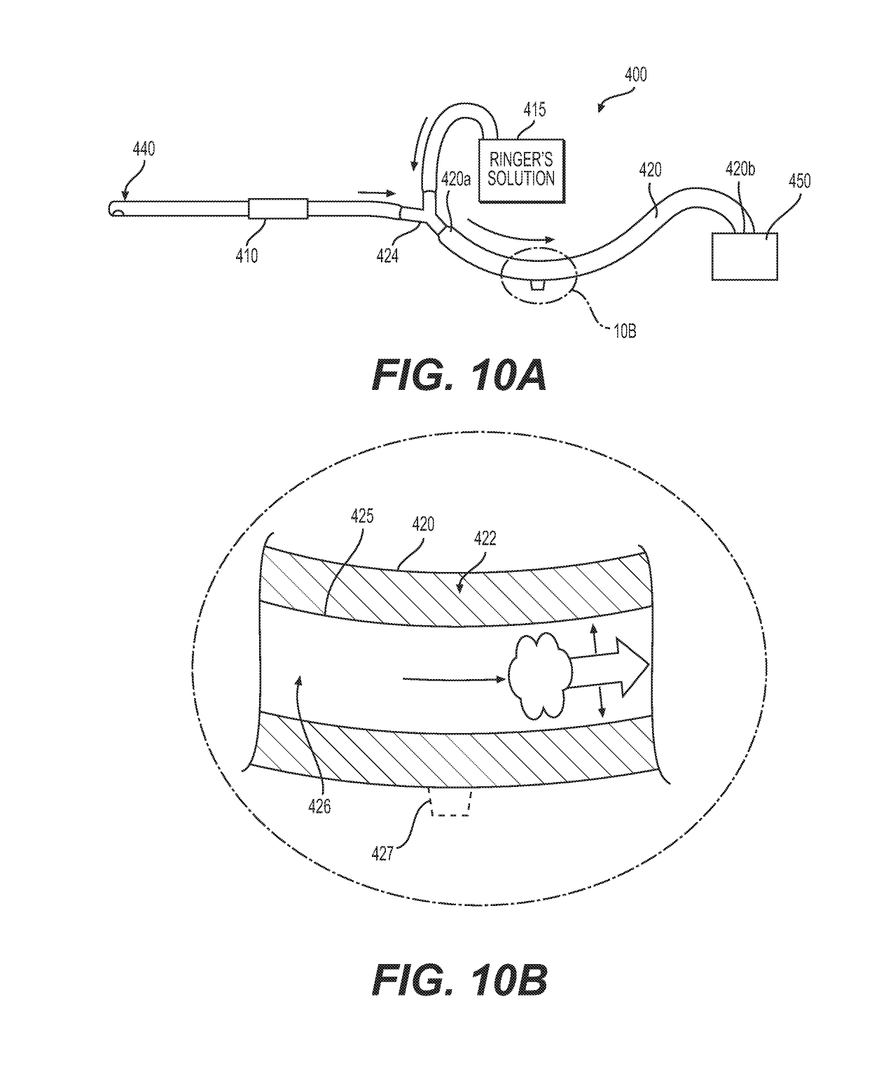

FIGS. 10A and 10B illustrate a tubing system 400 for treating tissue in accordance with various embodiments of the present disclosure. The tubing system 400 can include a tube section 420 with a filter portion 425 including a lumen 426 passing therethrough. The filter portion 425 can be surrounded by an absorbent material 422. As fluid or tissue flows through the lumen 426 of the filter portion 425, unwanted waste components such as blood, fats, or other material can pass through pores in the filter portion 425 and become absorbed within the absorbent material 422. The tubing system 400 streamlines tissue harvesting and processing by eliminating transitions between stages of harvest, wash, and waste separation by providing a continuous flow from harvest to filling of injection devices.

In some embodiments, the tubing system 400 can be attached to one or more of a harvest device 410 including a cannula 440, a wash fluid reservoir 415 including a wash solution, or a tissue reservoir 450 that stores cleaned or processed tissue. In some embodiments, a first end 420a of the tubing system 400 can be in fluid communication with both the wash fluid reservoir 415 and the harvest device 410. For example, a Y-connector 424 can be used to connect the first end 420a to both the wash fluid reservoir 415 and the harvest device 410 at the same time. The connection can be facilitated by tubing, hosing, or piping. In some embodiments, a second end 420b of the tubing system 420 can be in fluid communication with the tissue reservoir 450. The connection between the tissue reservoir 450 and the tubing system 420 can be facilitated by tubing, hosing, or piping.

In accordance with various embodiments, the filter portion 425 can include a mesh wall or solid wall that includes holes or pores. In some embodiments, the filter portion 425 can be substantially similar to the filter 115 described above with reference to FIG. 1.

In some embodiments, the absorbent material 422 can include one or more of acrylic, alginate, hydrocolloid, cellulose, cloth, collagen, or other suitable materials alone or in combination. The absorbent material 422 can include a foam. In some embodiments, the absorbent material 422 can trap fluids and waste matter within itself. In some embodiments, the absorbent material 422 can create a wicking effect (e.g., caused by physical effects such as surface tension or osmosis) that draws wash solution and wastes from within the lumen 426 and into the material.

In some embodiments, the absorbent material 422 or filter portion 425 of the tubing system 420 can be removable or replaceable. For example, the absorbent material 422 or filter tube 425 can be replaced within a sterile field when not in use. By allowing for a removable or replaceable filter tube 425 or absorbent material 422, tubes with clogged pores or holes and absorbent material that is at or beyond its maximum absorption capacity can be swapped for new versions that allow for additional tissue collection in the same operation.

The tubing system can optionally include one or more ports 427 that pass through the outer wall and are in fluid communication with the absorbent material 422. The ports 427 can be connected to a negative pressure source such as a pump in some embodiments. Application of negative pressure to the port 427 can promote absorption of fluids from harvested tissue, increase the rate of absorption of fluids from the harvested tissue, and remove wastes from the absorbent material 422 to allow for further absorption beyond the maximum absorption capacity of the absorbent material 422.

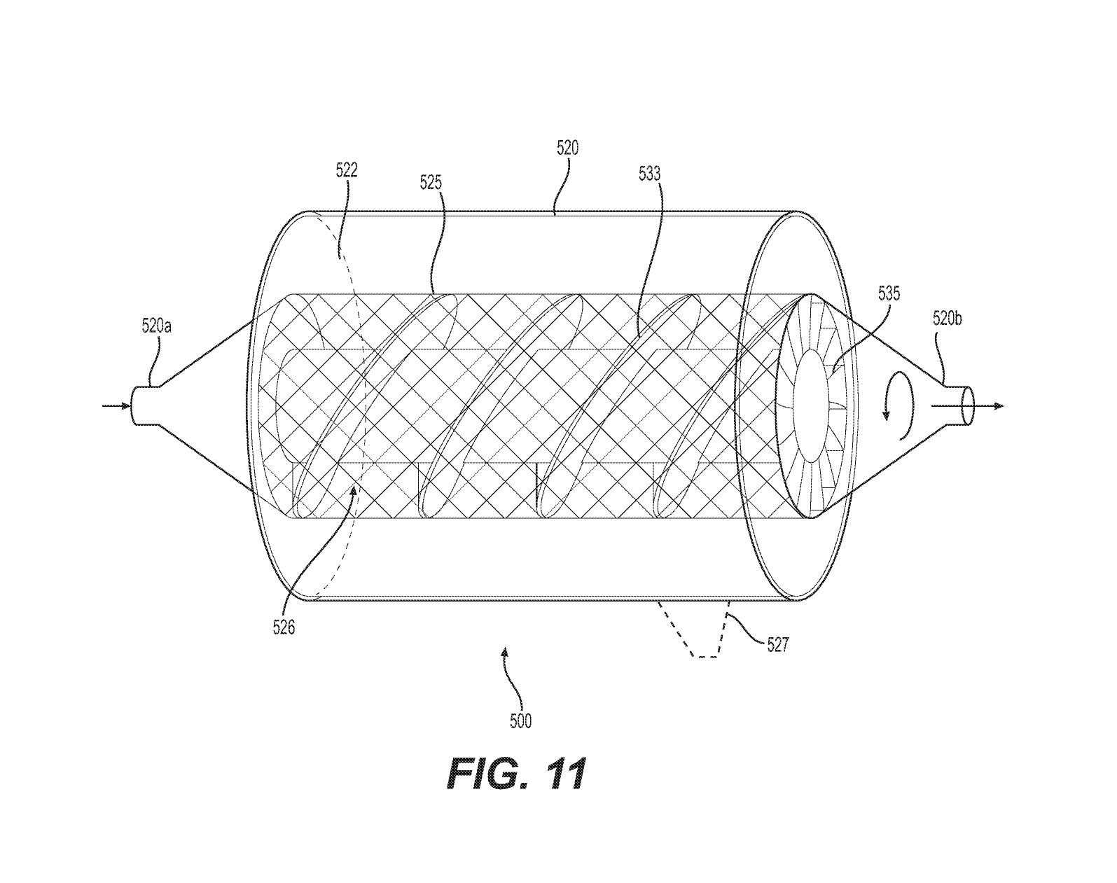

FIG. 11 illustrates an in-line system 500 for treating tissue including a turbine and an auger in accordance with various embodiments described herein. The system 500 can include an outer body 520 enclosing a volume and a filter 525 that divides the volume into an interior fill volume 526 and an exterior wash volume 522. Tissue to be processed can enter the system 500 at a first end 520a and progress through the interior fill volume 526. An auger 533 can cause the tissue to travel down the length of the fill volume 526 and can help to press the tissue against the filter 525. Waste and wash fluid can be pressed through the filter 525 and enter the exterior waste volume 522. The processed tissue can exit the system 500 at a second end 520b after passing through a turbine 535. The in-line system 500 allows tissue processing to occur simultaneously with tissue harvesting and reduces the time and number of steps typically needed to process tissue in conventional systems.

In some embodiments, the exterior wash volume 522 can include an absorbent material. As described above with reference to FIGS. 10A and 10B, the absorbent material can draw fluid and waste material through the filter and retain the waste or fluid within itself. In some embodiments, the outer body 520 can optionally include a port 527 connected to a source of negative pressure. The port 527 can draw fluid into the absorbent material through the filter 525 and can draw off excess fluids and waste material to allow the absorbent material to exceed its maximum absorption capacity.

In some embodiments, a vacuum or other source of negative pressure can be connected to the second end 520b of the device 500 to help draw tissue through the device. In addition, the application of negative pressure can simultaneously drive the turbine 535. In some embodiments, the turbine 535 can be operatively coupled to the auger 533 such that turning the turbine also powers motion of the auger to drive tissue through the interior fill volume 526.

Other embodiments will be apparent to those skilled in the art from consideration of the specification and practice of this disclosure. It is intended that the specification and examples be considered as exemplary only, with the true scope and spirit of the disclosed devices and methods being indicated by the following claims.

What is claimed is:

1. A system for treating tissue comprising:
a syringe body that encloses a volume;
a filter disposed in the syringe body and dividing the volume into an interior fill volume and an exterior wash volume, the filter configured to allow passage of fluids; and
a cannula coupled to the syringe body,
wherein applying a negative pressure to the syringe body draws a tissue through the cannula and into the interior fill volume and transfers fluid between the interior fill volume and the exterior wash volume.

2. The system of claim 1, further comprising a vacuum source couplable to the syringe body to apply the negative pressure.

3. The system of claim 2, wherein the vacuum source is portable.

4. The system of claim 2, wherein the vacuum source drains waste fluid from the exterior wash volume.

5. The system of claim 2, wherein the vacuum source is powered by a peristaltic pump.

6. The system of claim 1, further comprising a turbine drivable by application of negative pressure.

7. The system of claim 1, wherein the filter is a mesh.

8. The system of claim 7, wherein the mesh includes holes with a 200 micron diameter.

9. The system of claim 1, wherein the exterior wash volume includes an absorbent material.

10. The system of claim 1, wherein the interior fill volume includes an auger.

11. The system of claim 1, further comprising a syringe plunger to apply negative and positive pressure to the fill volume.

12. The system of claim 11, further comprising a valve to control fluid communication between the cannula and the syringe body.

13. The system of claim 1, further comprising at least one of a wash fluid reservoir or waste reservoir in fluid communication with the exterior wash volume.

14. The system of claim 1, wherein the enclosed volume in the syringe body is less than 100 mL.

15. A method for treating tissue comprising:
selecting a syringe body that encloses a volume and a filter disposed in the syringe body and dividing the volume into an interior fill volume and an exterior wash volume, the filter configured to allow passage of fluids, and a cannula coupled to the syringe body;
drawing a tissue through the cannula and into the interior fill volume by applying a negative pressure; and
transferring fluid from the tissue through the filter to the exterior wash volume by applying the negative pressure.

16. The method of claim 15, further comprising coupling a vacuum source to the syringe body to apply the negative pressure.

17. The method of claim 16, further comprising draining waste fluid from the exterior wash volume using the vacuum source.

18. The method of claim 15, further comprising driving a turbine by application of negative pressure.

19. The method of claim 15, further comprising absorbing fluid in the exterior wash volume using an absorbent material.

20. The method of claim 15, further comprising transferring tissue in the interior fill volume using an auger.

21. The method of claim 15, wherein applying negative pressure includes operating a syringe plunger.

22. The method of claim 15, further comprising injecting the tissue at an implantation site.
